(19) United States
(12) Patent Application Publication
(10) Pub. No.: US 2023/0038182 A1
(43) Pub. Date: Feb. 9, 2023

(54) USER REMOVABLE FILL CLOSURE TAB WITH INTEGRATED MEMBRANE

(71) Applicant: Becton, Dickinson and Company, Franklin Lakes, NJ (US)

(72) Inventors: Nitish Kumar Varma KUNAPARAJU, Burlington, MA (US); Alexander John SEGIT, Eliot, ME (US); Alessandro E. PIZZOCHERO, Chelmsford, MA (US)

(21) Appl. No.: 17/808,427

(22) Filed: Jun. 23, 2022

Related U.S. Application Data
(60) Provisional application No. 63/214,550, filed on Jun. 24, 2021.

(51) Int. Cl. A61M 5/142

(52) U.S. Cl. CPC A61M 5/14248; A61M 2005/1585; A61M 2005/14252

(57) ABSTRACT

Provided is a fill closure tab or an infusion device incorporating the same. The fill closure tab includes a gripping portion, and seals an insertion mechanism opening with a hydrophobic membrane. The fill closure tab further includes a fill assist mechanism shaped to assist with directing a filling device such as a pen needle or syringe to a fill port of the infusion device.

### RELATED APPLICATION

The present application claims priority to U.S. Provisional Application No. 63/214,550, filed on Jun. 24, 2021, including the specification, drawings and abstract, the entire disclosure of which is incorporated herein by reference.

### FIELD OF THE INVENTION

The present invention relates generally to medical infusion systems. More particularly, the present invention relates to a fill closure tab for improving the filling of medical infusion systems with medication.

### BACKGROUND OF THE INVENTION

Diabetes is a group of diseases characterized by high levels of blood glucose resulting from the inability of diabetic patients to maintain proper levels of insulin production when required. Persons with diabetes will require some form of daily insulin therapy to maintain control of their glucose levels. Diabetes can be dangerous to the affected patient if it is not treated, and it can lead to serious health complications and premature death. However, such complications can be minimized by utilizing one or more treatment options to help control the diabetes and reduce the risk of complications.

The treatment options for diabetic patients include specialized diets, oral medications and/or insulin therapy. The main goal of diabetes treatment is to control the diabetic patient's blood glucose or sugar level. However, maintaining proper diabetes management may be complicated because it has to be balanced with the activities of the diabetic patient.

For the treatment of type 1 diabetes, there are two principal methods of daily insulin therapy. In the first method, diabetic patients use syringes or insulin pens to self-inject insulin when needed. This method requires a needle stick for each injection, and the diabetic patient may require three to four injections daily. The syringes and insulin pens that are used to inject insulin are relatively simple to use and cost effective.

Another effective method for insulin therapy and managing diabetes is infusion therapy or infusion pump therapy in which an insulin pump is used. The insulin pump can provide continuous infusion of insulin to a diabetic patient at varying rates in order to more closely match the functions and behavior of a properly operating pancreas of a non-diabetic person that produces the required insulin, and the insulin pump can help the diabetic patient maintain his/her blood glucose level within target ranges based on the diabetic patient's individual needs.

Infusion pump therapy requires an infusion cannula, typically in the form of an infusion needle or a flexible catheter, that pierces the diabetic patient's skin and through which, infusion of insulin takes place. Infusion pump therapy offers the advantages of continuous infusion of insulin, precision dosing, and programmable delivery schedules.

In infusion therapy, insulin doses are typically administered at a basal rate and in a bolus dose. When insulin is administered at a basal rate, insulin is delivered continuously over 24 hours in order to maintain the diabetic patient's blood glucose levels in a consistent range between meals and rest, typically at nighttime. Insulin pumps may also be capable of programming the basal rate of insulin to vary according to the different times of the day and night. In contrast, a bolus dose is typically administered when a diabetic patient consumes a meal, and generally provides a single additional insulin injection to balance the consumed carbohydrates. Insulin pumps may be configured to enable the diabetic patient to program the volume of the bolus dose in accordance with the size or type of the meal that is consumed by the diabetic patient. In addition, insulin pumps may also be configured to enable the diabetic patient to infuse a correctional or supplemental bolus dose of insulin to compensate for a low blood glucose level at the time when the diabetic patient is calculating the bolus dose for a particular meal that is to be consumed.

Insulin pumps advantageously deliver insulin over time rather than in single injections, typically resulting in less variation within the blood glucose range that is recommended. In addition, insulin pumps may reduce the number of needle sticks which the diabetic patient must endure, and improve diabetes management to enhance the diabetic patient's quality of life.

Typically, regardless of whether a diabetic patient uses multiple direct injections (MDIs) or a pump, the diabetic patient takes fasting blood glucose medication (FBGM) upon awakening from sleep, and also tests for glucose in the blood during or after each meal to determine whether a correction dose is required. In addition, the diabetic patient may test for glucose in the blood prior to sleeping to determine whether a correction dose is required, for instance, after eating a snack before sleeping.

To facilitate infusion therapy, there are generally two types of insulin pumps, namely, conventional pumps and patch pumps. Conventional pumps require the use of a disposable component, typically referred to as an infusion set, tubing set or pump set, which conveys the insulin from a reservoir within the pump into the skin of the user. The infusion set consists of a pump connector, a length of tubing, and a hub or base from which a cannula, in the form of a hollow metal infusion needle or flexible plastic catheter extends. The base typically has an adhesive that retains the base on the skin surface during use. The cannula can be inserted onto the skin manually or with the aid of a manual or automatic insertion device. The insertion device may be a separate unit required by the user.

Another type of insulin pump is a patch pump. Unlike a conventional infusion pump and infusion set combination, a patch pump is an integrated device that combines most or all of the fluidic components, including the fluid reservoir, pumping mechanism and mechanism for automatically inserting the cannula, in a single housing which is adhesively attached to an infusion site on the patient's skin, and does not require the use of a separate infusion or tubing set. A patch pump containing insulin adheres to the skin and delivers the insulin over a period of time via an integrated subcutaneous cannula. Some patch pumps may wirelessly communicate with a separate controller device (as in one device sold by Insulet Corporation under the brand name OmniPod®), while others are completely self-contained. Such devices are replaced on a frequent basis, such as every three days, when the insulin reservoir is exhausted or complications may otherwise occur, such as restriction in the cannula or the infusion site.

As patch pumps are designed to be a self-contained unit that is worn by the diabetic patient, it is preferable to be as small as possible so that it does not interfere with the activities of the user. Thus, in order to minimize discomfort to the user, it would be preferable to minimize the overall thickness of the patch pump. However, in order to minimize the thickness of the patch pump, its constituent parts should be reduced as much as possible. One such part is the insertion mechanism for inserting the cannula into the user's skin.

Since patch pumps are typically provided to patients empty, they require a means of filling the patch pump with medication. Some users have low dexterity, and accordingly medication fill systems should be as easy as possible for users to operate. In addition, fill systems should minimize any adverse effects of overfilling the patch pump with medication.

Accordingly, a need exists for an improved fill system for use in a patch pump or other infusion device. Such a fill system preferably operates in conjunction with a cannula insertion mechanism of the patch pump or other infusion device.

### SUMMARY OF THE INVENTION

The above described needs are addressed and other advantage realized with a fill closure tab for an infusion device. The fill closure tab includes a gripping portion, an attachment portion shaped to attach the fill closure tab to an infusion device, and a hydrophobic membrane arranged to cover a needle opening on a bottom surface of the infusion device. The fill closure tab is preferably removable from the infusion device.

Additional and/or other aspects and advantages of the present invention will be set for in the description that follows, or will be apparent from the description, or may be learned by the practice of the invention. The present invention may comprise a method or apparatus or system having one or more of the above aspects, and/or one or more of the features and combinations thereof. The present invention may comprise one or more of the features and/or combinations of the above aspects as recited, for example, in the attached claims.

### BRIEF DESCRIPTION OF THE DRAWINGS

The various objects, advantages and novel features of the exemplary embodiments of the present invention will be more readily appreciated from the following detailed description when read in conjunction with the appended drawings, in which:

FIG. 1 is an isometric view of an exemplary insertion device in a pre-activation state in accordance with an embodiment of the present invention;

FIG. 2 is another isometric view of the insertion device of FIG. 1 in a pre-activation state in accordance with an embodiment of the present invention;

FIG. 3 is a view of the insertion device of FIG. 1 in a post-activation state in accordance with an embodiment of the present invention;

FIG. 4 is an exploded view of the insertion device of FIG. 1 in accordance with an embodiment of the present invention;

FIG. 5 is a sectional view of a catheter/septum subassembly of the insertion device of FIG. 1 in accordance with an embodiment of the present invention;

FIG. 6 is a view of the assembly of the button subassembly and springs into the housing of the insertion device of FIG. 1 and illustrating the use of temporary protective tubing on the catheter in accordance with an embodiment of the present invention;

FIG. 7 is a view of the partially complete assembly of the button subassembly and springs into the housing of the insertion device of FIG. 1 and illustrating the use of temporary protective tubing on the catheter in accordance with an embodiment of the present invention;

FIG. 8 is a perspective view of a patch pump incorporating a low-profile cannula insertion device, illustrated without a cover for clarity;

FIG. 9 is an exploded view of the various components of the patch pump of FIG. 8, illustrated with a cover;

FIG. 10 is a perspective view of an alternative design for a patch pump having a flexible reservoir, illustrated without a cover;

FIG. 11 is a patch-pump fluidic architecture and metering sub-system diagram of the patch pump of FIG. 10;

FIGS. 12-15 are cross sectional views of an insertion mechanism according to an embodiment of the present invention;

FIG. 16 illustrates a septum according to an embodiment of the present invention;

FIGS. 17-19 illustrate another embodiment of a fill tab according to an embodiment of the present invention;

FIG. 20 illustrates a side elevation view of the fill tab of FIGS. 17-19;

FIGS. 21A-21C illustrate alternative embodiments of the fill tab;

FIGS. 22A-22B illustrate a pen needle connected to a patch pump according to an embodiment of the present invention;

FIGS. 23A-23B illustrate a syringe connected to a patch pump according to an embodiment of the present invention;

FIGS. 24A and 24B illustrate an embodiment of the present invention including an insertion button safety cover;

FIG. 25 illustrates an adhesive patch on a bottom surface of a patch pump according to an embodiment of the present invention;

FIGS. 26-31 illustrate an embodiment of the present invention including a fill tubing extending from a bottom surface of the patch pump; and

FIGS. 32A-32B illustrate a fill tab according to another mbodiment of the present invention.

Throughout the drawings, like reference numerals will be understood to refer to like parts, components and structures.

### DETAILED DESCRIPTION OF THE EXEMPLARY EMBODIMENTS

The exemplary embodiments of the present invention described below provide novel means of providing one or more infusion device elements that are configured to insert catheter up to 8 mm into a skin surface, but embodiments are not limited thereto. The insertion device is configured to perform a manual insertion of the catheter which allows the insertion device to be smaller, simpler and cheaper than automatic or spring-assisted insertion devices.

Exemplary embodiments of the present invention described below, utilize a manual insertion device and include a dual retraction spring configuration for automatic introducer needle retraction that also allows for a very small device size. The dual retraction spring configuration is implemented using a plurality of cylindrical or barrel-shaped guides. In an exemplary embodiment, one barrel guides a button and catheter, and adjacent barrels house retraction springs, one on each side of the button and catheter. Having the springs in separate barrels allows for much smaller springs than a single-barrel configuration in which the spring is coaxial with the catheter. A single coaxial spring creates access to the button assembly since spring design limitations require the spring to extend nearly from the bottom of the housing to the top. Access is required for features like the locking arm and if the features are implemented inside the spring, the entire mechanism must grow to accommodate them increasing the mechanism foot print.

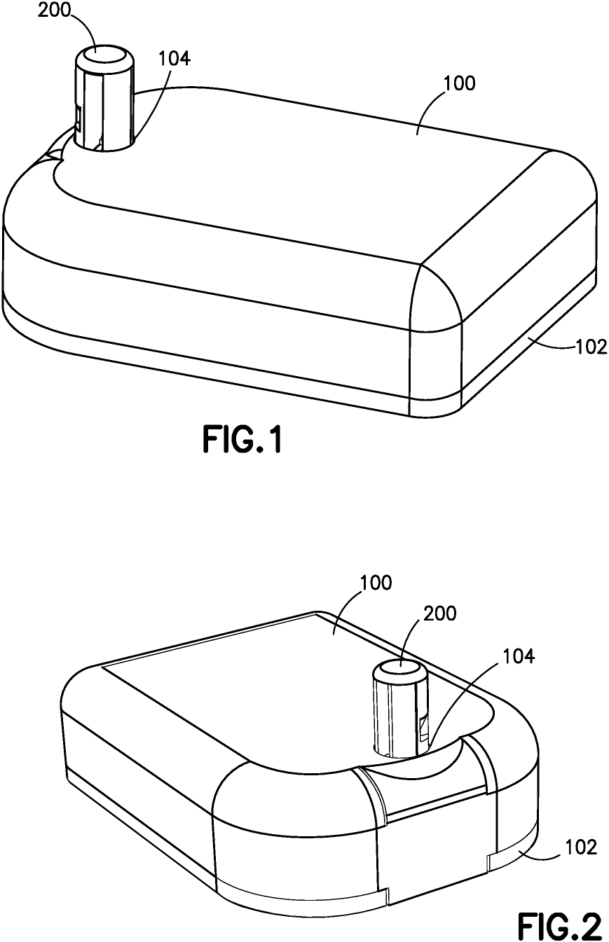

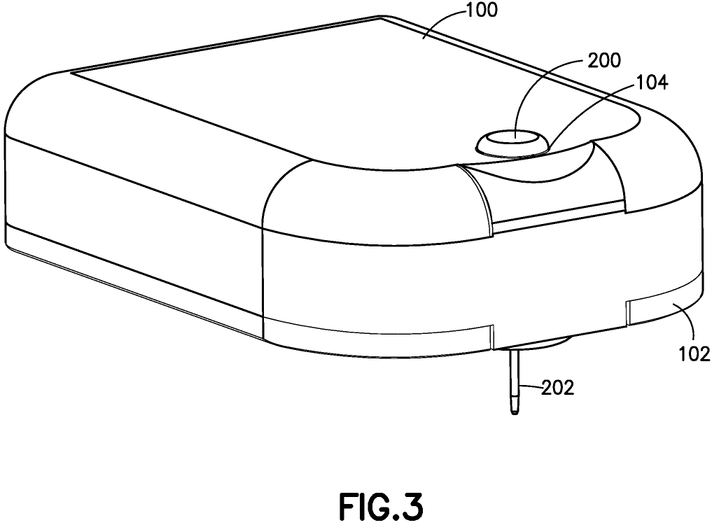

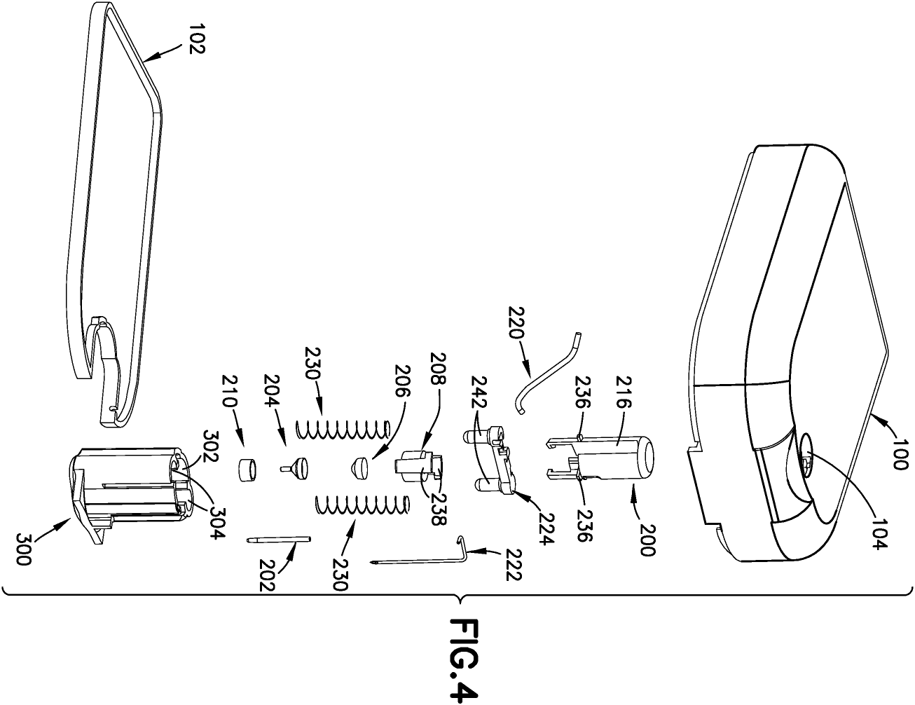

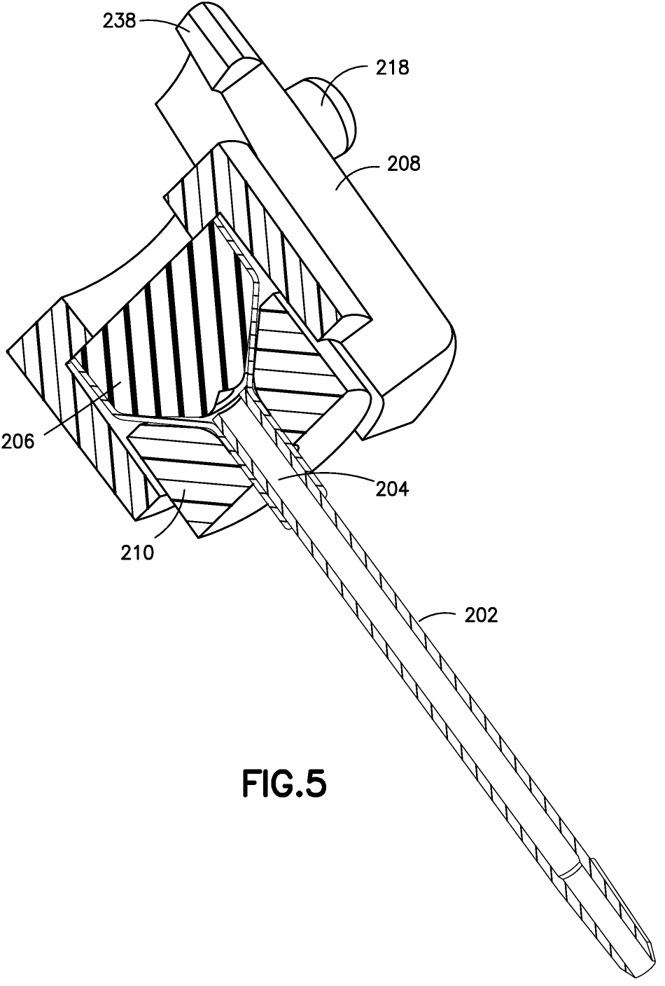

FIGS. 1 and 2 show the insertion device before use and FIG. 3 shows the device after deployment of the cannula. As shown in FIGS. 1-3, the insertion device includes a top housing 100 and a base 102. The top housing 100 is shown having an opening 104 through a top surface from which a user-accessible, and user-acutatable button 200 slidably extends. The content of the insertion device, including the mechanism housing 300, is shown in greater detail in FIG. 4. The top housing 100, button 200, and mechanism housing 300 can be manufactured from ABS, and the base 102 can be manufactured from PETG, but embodiments are not limited thereto.

As shown in FIG. 4, the exemplary insertion device is assembled by stacking together a number of subassemblies which are trapped between the top housing 100 and the mechanism housing 300. FIG. 4 is a view of the insertion device of FIG. 1 in accordance with an embodiment of the present invention. The subassemblies of FIG. 4 and discussed in greater detail below include a catheter/septum subassembly, an introducer needle subassembly, and a button subassembly. Other features and functions of the insertion device that are well-known to those skilled in the art are omitted from the figures and discussion for clarity.

An exemplary catheter/septum subassembly is shown in FIG. 5. FIG. 5 is a sectional view of a catheter/septum subassembly of the insertion device of FIG. 1 in accordance with an embodiment of the present invention. As shown in FIG. 5, the catheter/septum subassembly is assembled by attaching a catheter 202 on a metal wedge 204, then inserting a septum 206 in the wedge and trapping it between a release collar 208 and a catheter wedge cap 210. The septum 206 is radially compressed by the wedge 204 and axially compressed by the release collar 208 to create a seal between the septum 206 and wedge 204. The catheter 202 can be a 24G plastic catheter manufactured using FEP, and the release collar 208 and catheter wedge cap 210 can be manufactured using PTEG, but embodiments are not limited thereto. The wedge 204 can be manufactured using 305 stainless steel, and the septum 206 can be manufactured using isoprene, but embodiments are not limited thereto.

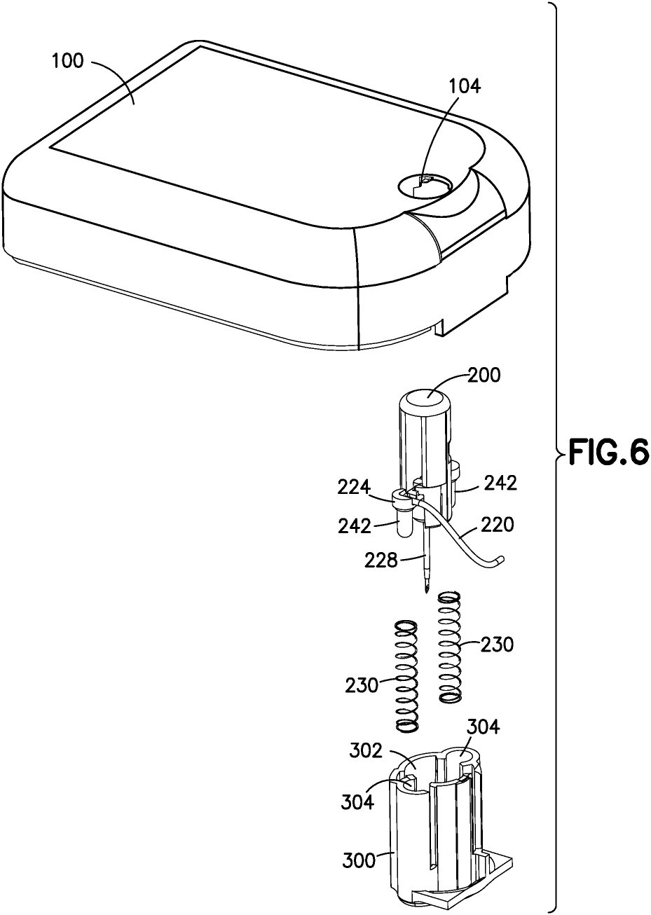

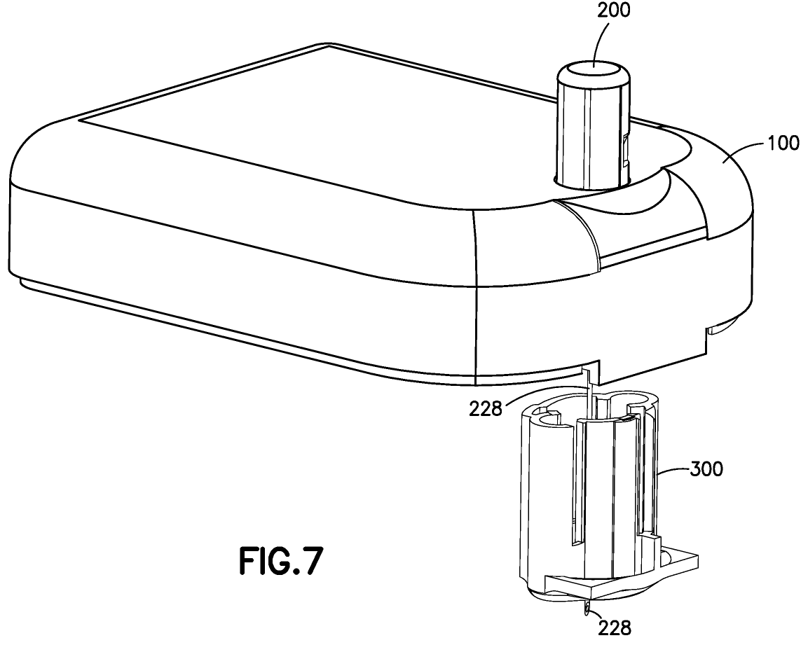

FIG. 6 is a view of the assembly of the button subassembly and springs into the housing of the insertion device of FIG. 1 and illustrating the use of temporary protective tubing on the catheter, and FIG. 7 is a view of the partially complete assembly of the button subassembly and springs into the housing of the insertion device of FIG. 1.

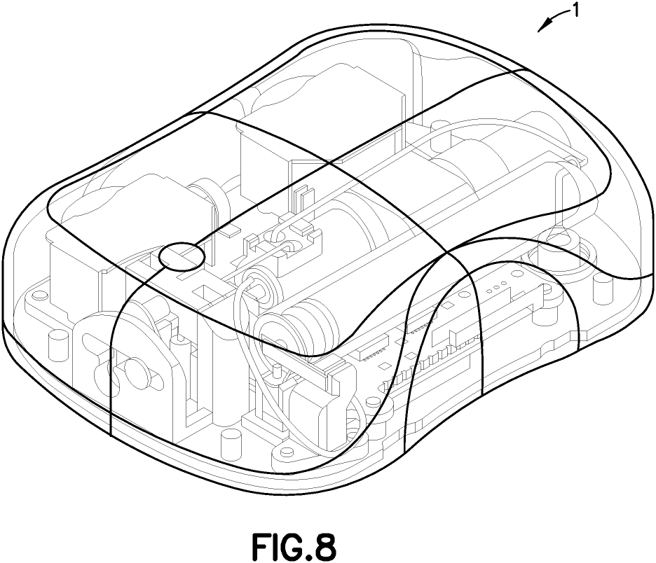

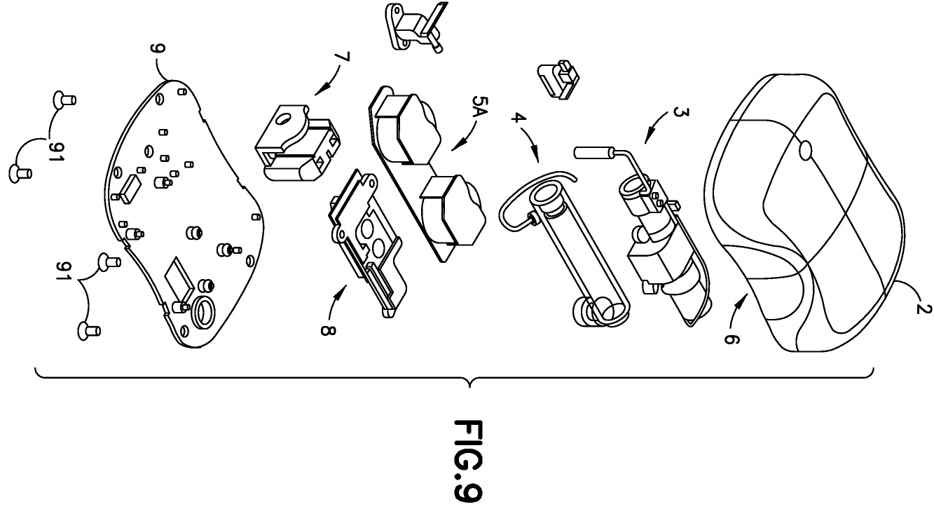

In the above embodiments, a patch pump can be provided with one or more of the described features. FIG. 8 is a perspective view of an exemplary embodiment of a patch pump 1 according to an exemplary embodiment of the invention. The patch pump 1 is illustrated with a see-through cover for clarity and illustrates various components that are assembled to form the patch pump 1. FIG. 9 is a view of the various components of the patch pump of FIG. 8, illustrated with a solid cover 2. The various components of the patch pump 1 may include: a reservoir 4 for storing insulin; a pump 3 for pumping insulin out of the reservoir 4; a power source 5 in the form of one or more batteries; an insertion mechanism 7 for inserting an inserter needle with a catheter into a user's skin; control electronics 8 in the form of a circuit board with optional communications capabilities to outside devices such as a remote controller and computer, including a smart phone; a dose button 6 on the cover 2 for actuating an insulin dose, including a bolus dose; and a base 9 to which various components above may be attached via fasteners 91. The patch pump 1 also includes various fluid connector lines that transfer insulin pumped out of the reservoir 4 to the infusion site.

As noted above, it should be understood that inserter mechanisms come in various configurations. In some embodiments, the inserter mechanism inserts a soft catheter into the skin. In these embodiments, typically the soft catheter is supported on a rigid insertion needle. The insertion needle is inserted into the skin along with the soft catheter, and then retracted from the skin, leaving the soft catheter in the skin. In other embodiments, a soft catheter is not provided, and the insertion needle remains in the skin and forms a portion of the insulin flow path to deliver insulin until the infusion is finished. Insertion needles are typically hollow, and need to be hollow if they form part of the insulin flow path. However, insertion needles that support a soft catheter and then retract may be solid or hollow. If the insertion needle deploys a soft catheter, and retracts but remains part of the insulin flow path, then the insertion needle should be hollow. However, if the insertion needle deploys a soft catheter and then retracts but does not form part of the insulin flow path, then the insertion needle may be solid or hollow. In either case, the insertion needle is preferably rigid enough to reliably penetrate the skin, but otherwise may be made flexible enough to provide comfort to the user.

FIG. 10 is a perspective view of an alternative design for a patch pump 1A having a flexible reservoir 4A, and illustrated without a cover. Such arrangement may further reduce the external dimensions of the patch pump 1A, with the flexible reservoir 4A filling voids within the patch pump 1A. The patch pump 1A is illustrated with a conventional cannula insertion device 7A that inserts the cannula, typically at an acute angle, less than 90 degrees, at the surface of a user's skin. The patch pump 1A further comprises: a power source 5A in the form of batteries; a metering sub-system 41 that monitors the volume of insulin and includes a low volume detecting ability; control electronics 8A for controlling the components of the device; and a reservoir fill port 43 for receiving a refill syringe 45 to fill the reservoir 4A.

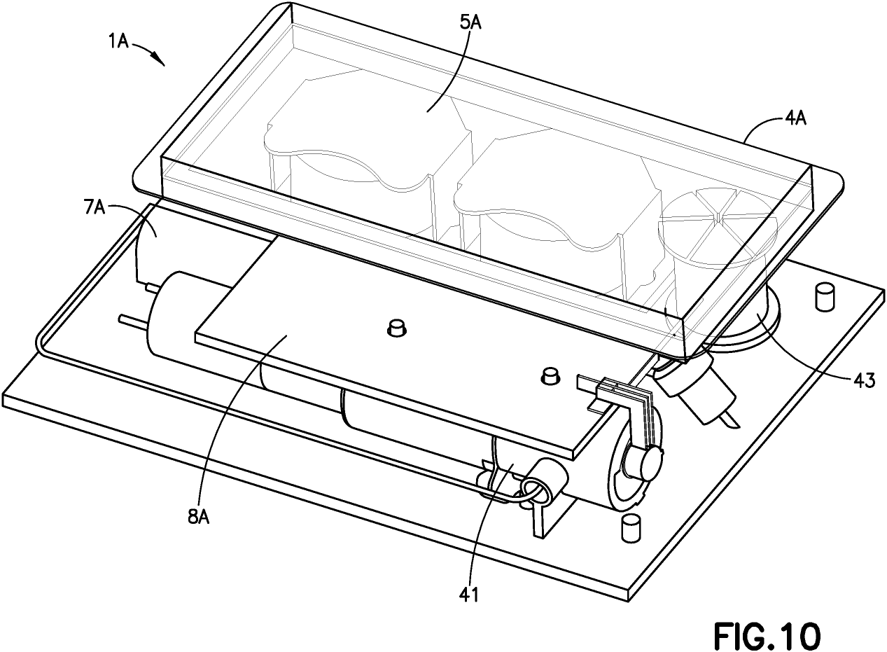

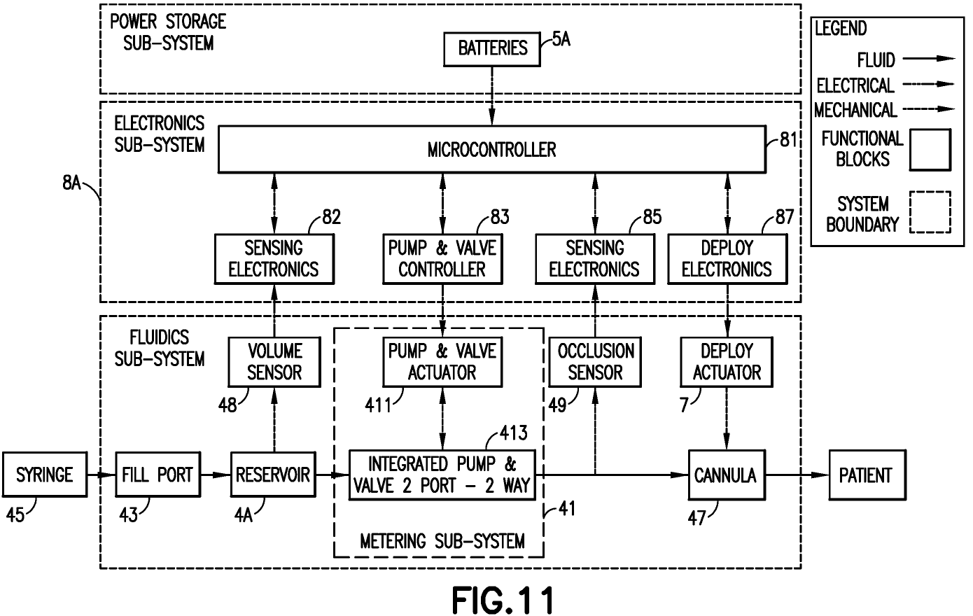

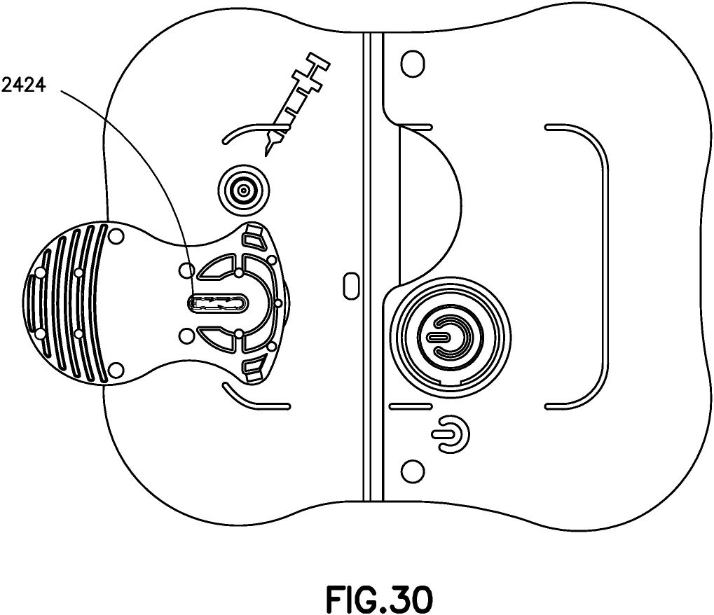

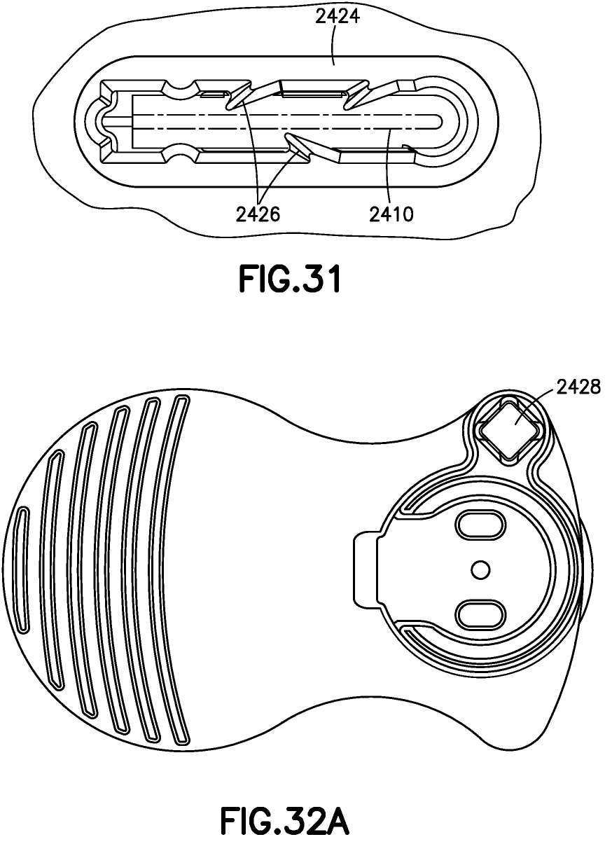

FIG. 11 is a patch-pump fluidic architecture and metering sub-system diagram of the patch pump 1A of FIG. 10. The power storage sub-system for the patch pump 1A includes batteries 5A. The control electronics 8A of the patch pump 1A may include a microcontroller 81, sensing electronics 82, pump and valve controller 83, sensing electronics 85, and deployment electronics 87 that control the actuation of the patch pump 1A. The patch pump 1A includes a fluidics sub-system that may include a reservoir 4A, volume sensor 48 for the reservoir 4A, a reservoir fill port 43 for receiving a refill syringe 45 to refill the reservoir 4A. The fluidics sub-system may include a metering system comprising a pump and valve actuator 411 and an integrated pump and valve mechanism 413. The fluidics sub-system may further include an occlusion sensor 49, a deploy actuator 7, as well as the cannula 47 for insertion into an infusion site on the user's skin. The architecture for the patch pumps of FIGS. 30 and 31 is the same or similar to that which is illustrated in FIG. 33.

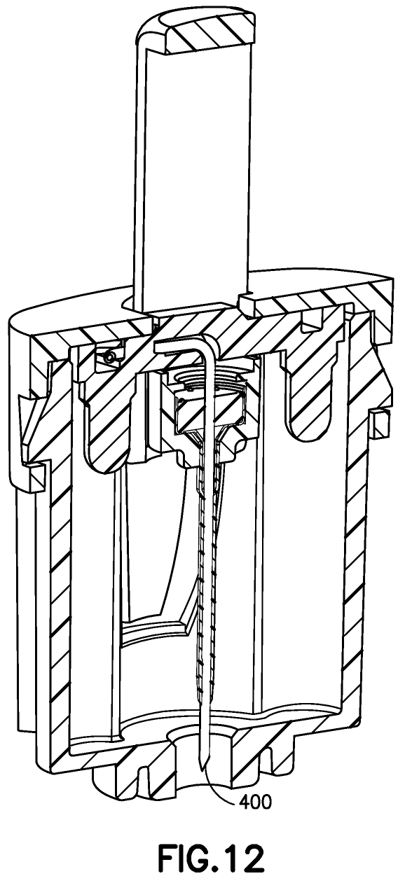

FIG. 12 illustrates an insertion mechanism similar in most respects to the insertion mechanisms discussed above. As illustrated, the needle tip 400 may be provided near an opening, such that the needle tip 400 is open to the environment. In such a scenario, when the user fills the device, it is possible that they overfill, and medicament will exit the cannula and leak out of the device.

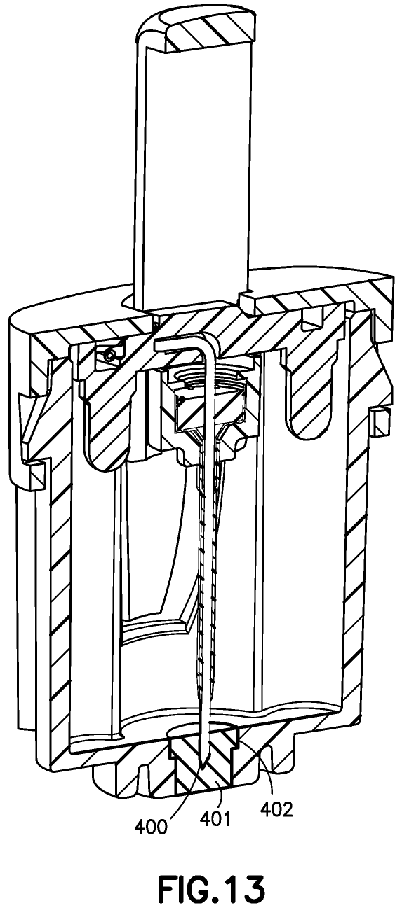

In an exemplary embodiment, as illustrated in FIG. 13, a permanent septum 401 is provided to seal the opening of the insertion mechanism through which the needle and cannula pass. The needle tip 400 is preferably embedded in the septum 401, thereby sealing the flow path and preventing leaks due to overfilling by a user. The septum 401 preferably includes a ridge 402 to prevent distal movement of the septum 401 during the insertion process.

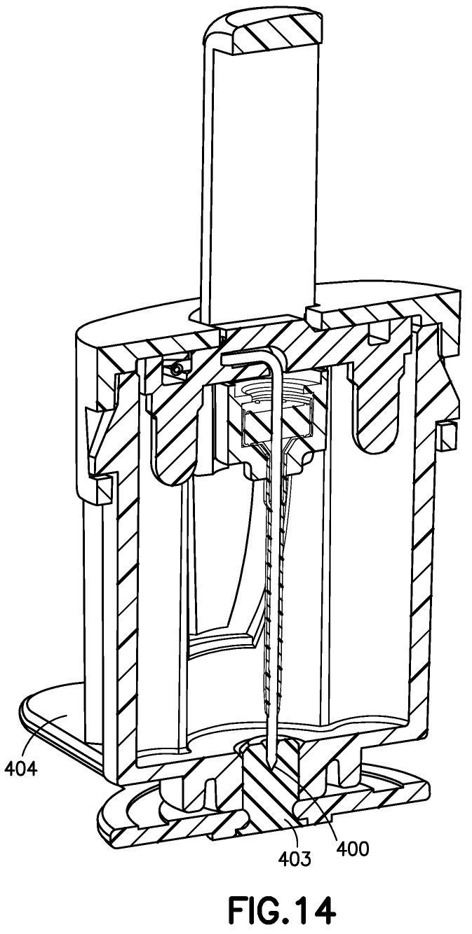

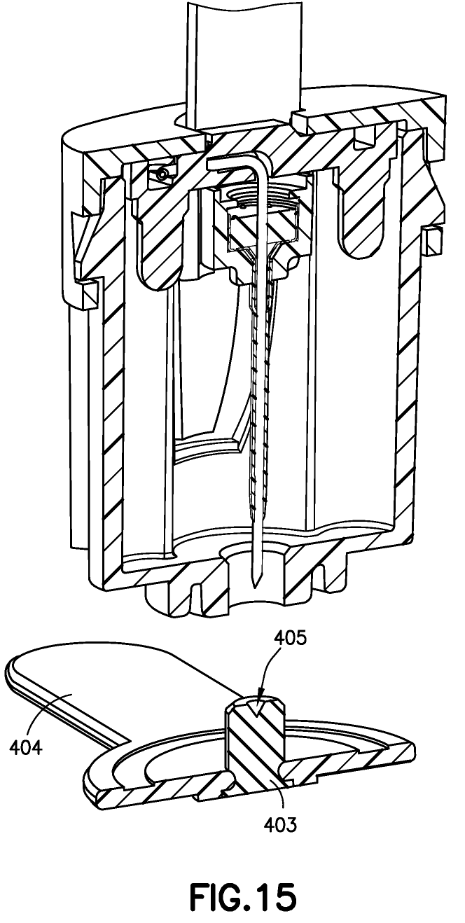

In another exemplary embodiment, as illustrated in FIG. 14, a removable septum 403 is provided, along with a tab 404 connected to the septum 403. In this embodiment, the needle tip 400 is still preferably embedded in the septum 403 before use. In order to use the device, the user fills the reservoir (not shown) while the tab 404 and removable septum 403 are in place, in order to prevent overfilling and/or leaks. FIG. 15 illustrates this embodiment in more detail. Preferably, the removable septum 403 is provided with an inwardly facing conical depression 405, which aids in keeping the needle and cannula concentric with the opening and septum 403. The conical depression 405 is also useful with the permanent septum illustrated in FIG. 16.

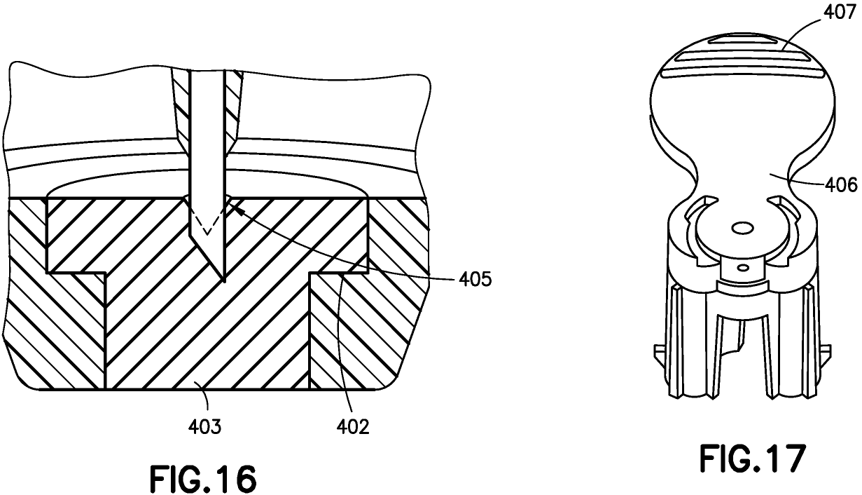

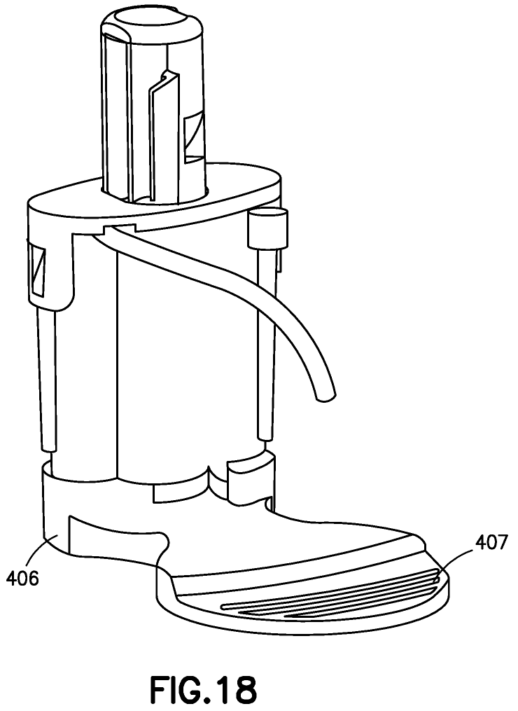

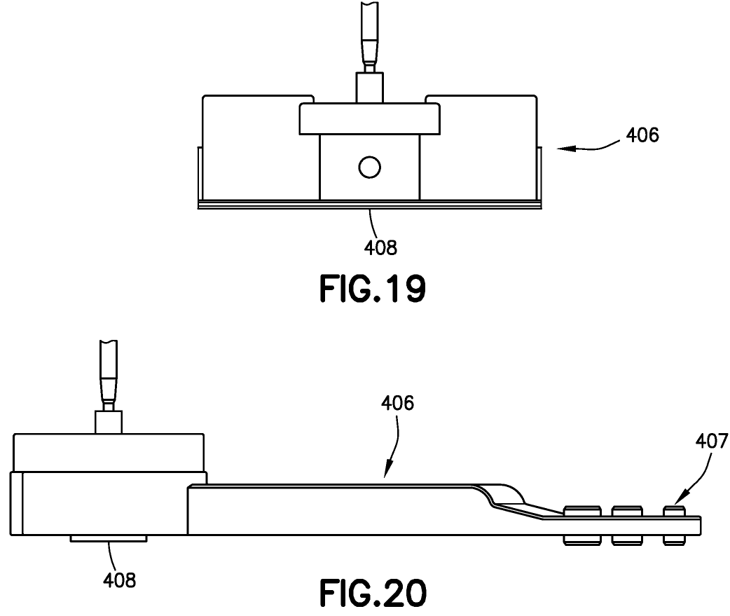

Another embodiment is illustrated in FIGS. 17-19. FIG. 17 is a bottom isometric view of a tab 406 that is shaped to attach to, and be detached from, an infusion device or patch pump, or the like. Tab 406 is preferably provided with a gripping portion 407 to make it easier to grab the tab and remove when appropriate. FIG. 18 illustrates the tab 406 from a top perspective view, and illustrates the insertion mechanism relative to the tab 406. The gripping portion 407 preferably is provided with ridges, or the like, to make it easier to grip. FIG. 19 is a side end view illustrating the tab 406. The tab 406 includes a hydrophobic membrane 408 that covers the opening of the insertion mechanism. Advantageously, the hydrophobic membrane 408 allows air to pass, thus relieving pressure during filling, but does not allow medicament to pass, thus preventing overfill and a leak. Moreover, this assists with priming the fluid pathway, and allows air to be removed. In some embodiments, the hydrophobic membrane 408 can be made to change color in the presence of medicament, thus providing a visual indication of fill or overfill. FIG. 20 is a side elevation view of the tab 406.

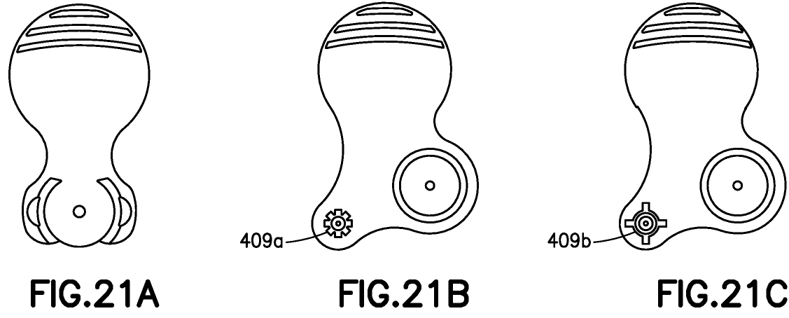

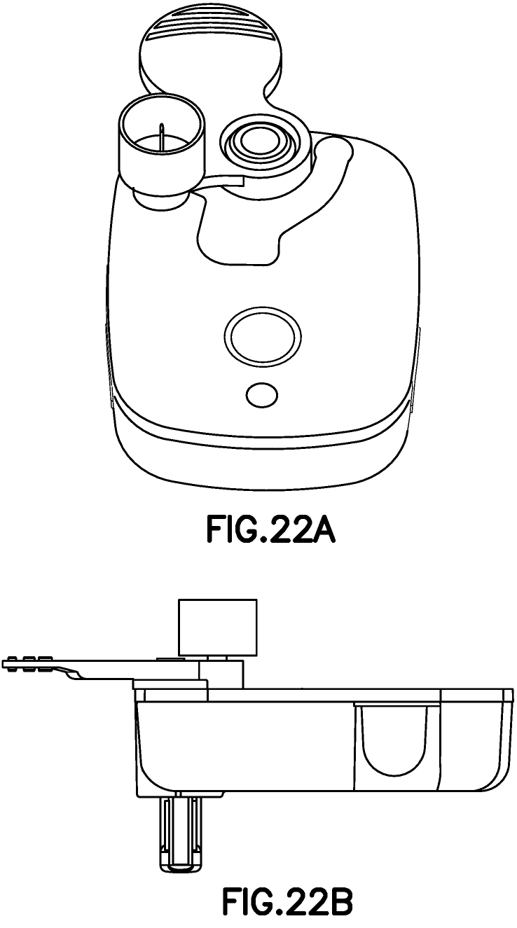

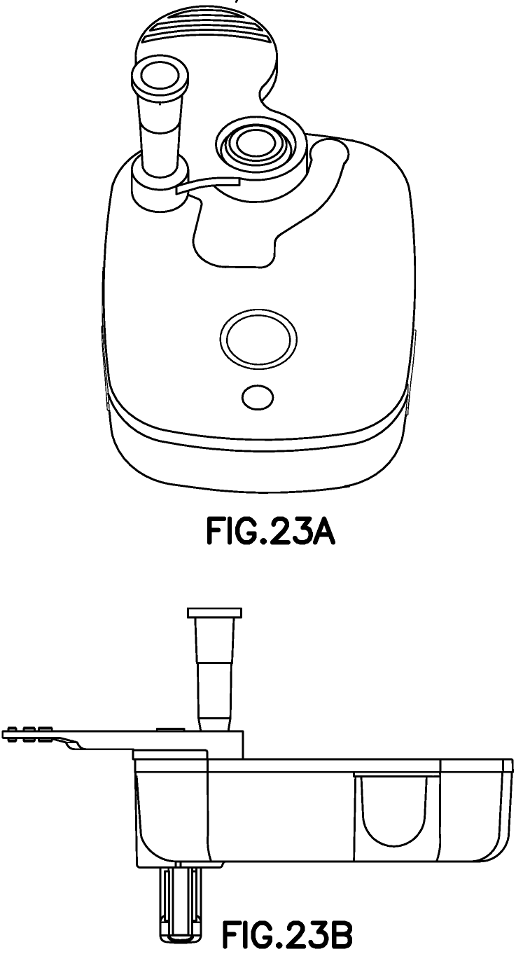

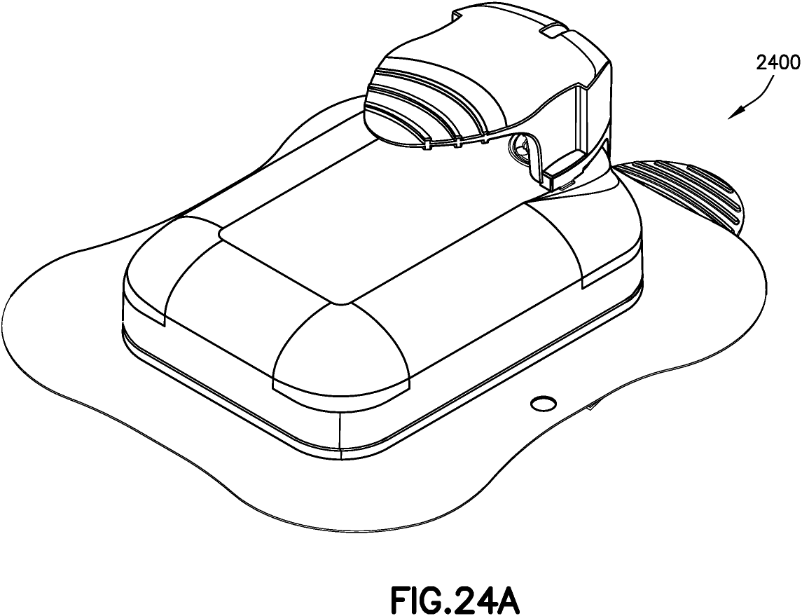

FIGS. 21A-21C illustrate alternative embodiments that can include a fill assist feature, as will now be described. The embodiment shown in FIG. 21A does not include a fill assist mechanism. The embodiment shown in FIG. 21B includes a fill assist mechanism 409a that is shaped to receive a pen needle, thus assisting a user to properly connect a pen needle to the fill port for filling of the device. FIG. 21C illustrates an embodiment having a fill assist mechanism 409b shaped to receive a syringe end. FIG. 22A and FIG. 22B illustrate the pen needle embodiment connected to a patch pump. FIGS. 23A and 23B illustrate the syringe embodiment connected to a patch pump. Either embodiment advantageously assists a user to aim a pen needle or syringe filled with medicament to the fill port of the patch pump for easier filling of the patch pump.

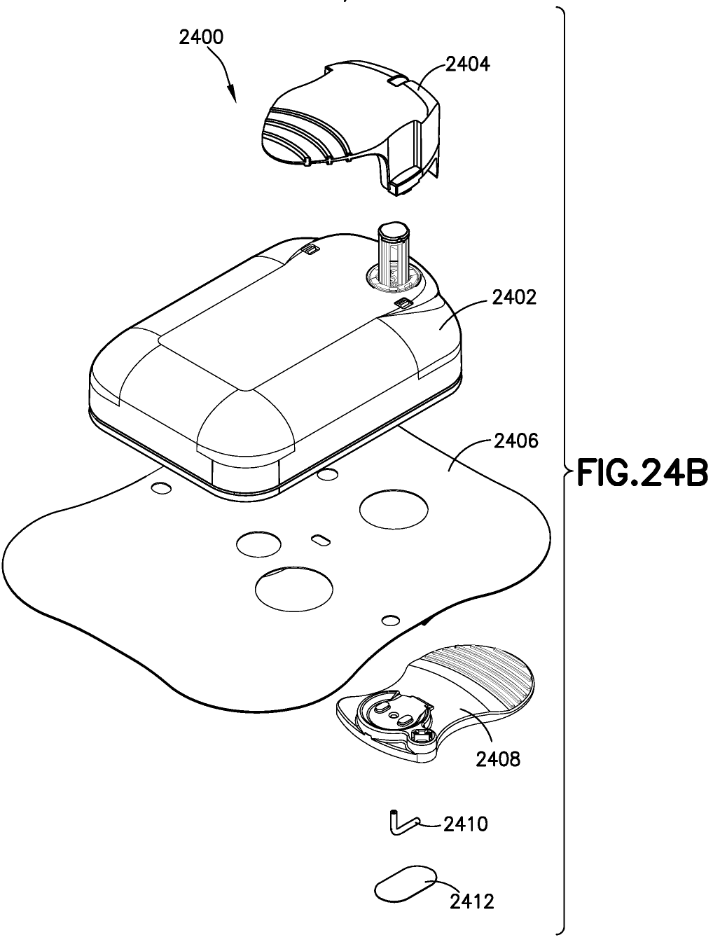

FIGS. 24A and 24B illustrate another embodiment of the present disclosure. Features that are substantially similar to previously described embodiments are omitted for conciseness and clarity. FIG. 24A is a perspective view of an IDD 2400 according to this embodiment and FIG. 24B is an exploded view of the same embodiment. The IDD 2400 comprises a patch pump enclosure 2402, an insertion button safety cover 2404, adhesive patch 2406, a fill tab 2408, fill tubing 2410 and a sealing member 2412.

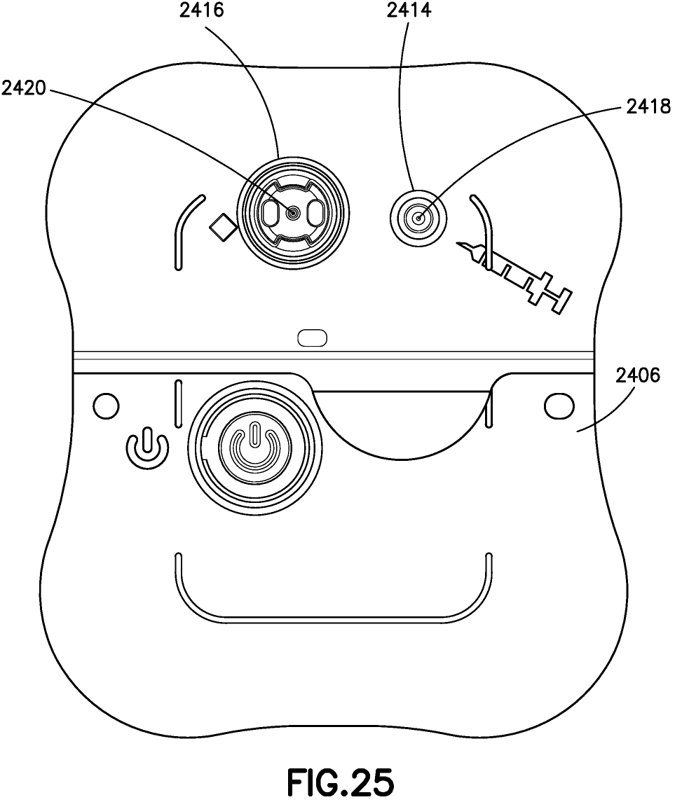

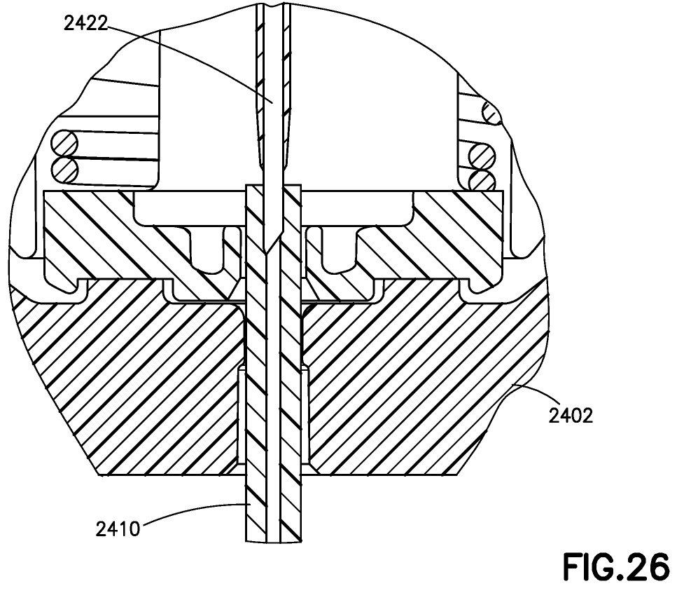

As illustrated in FIG. 25, adhesive patch 2406 includes holes 2414 and 2416 to expose the bottom surface of patch pump enclosure 2402 to provide access to a fill port 2418 and a catheter and insertion needle opening 2420.

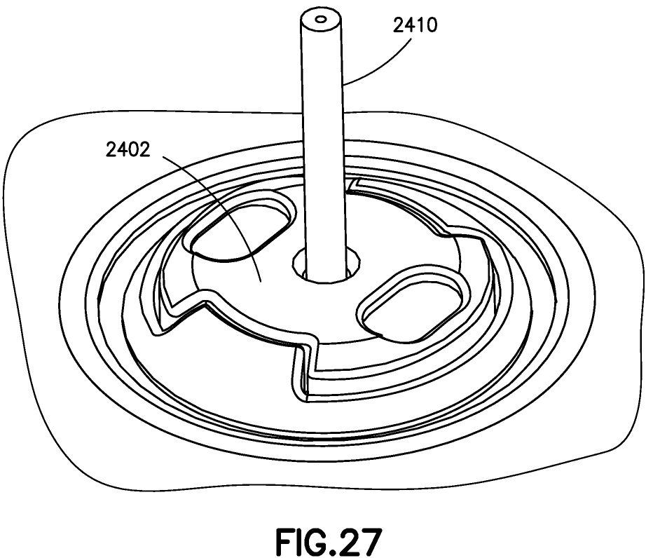

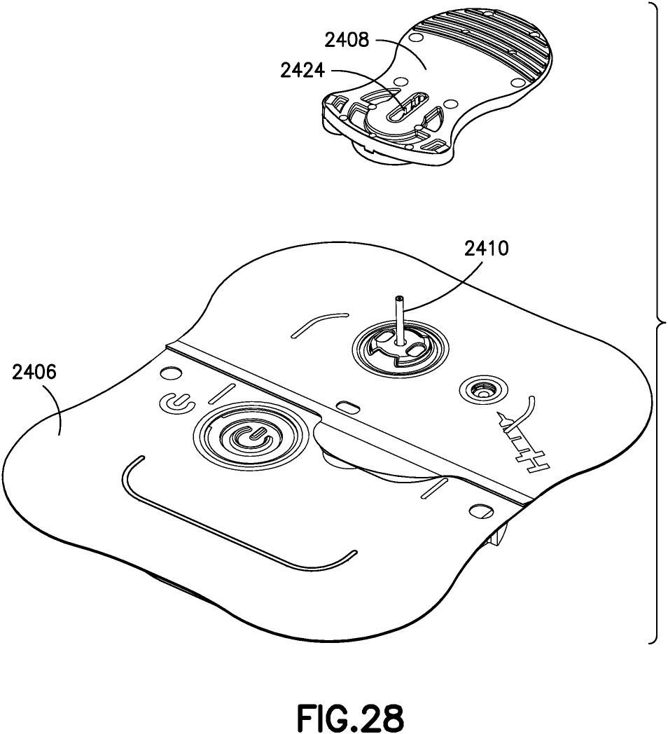

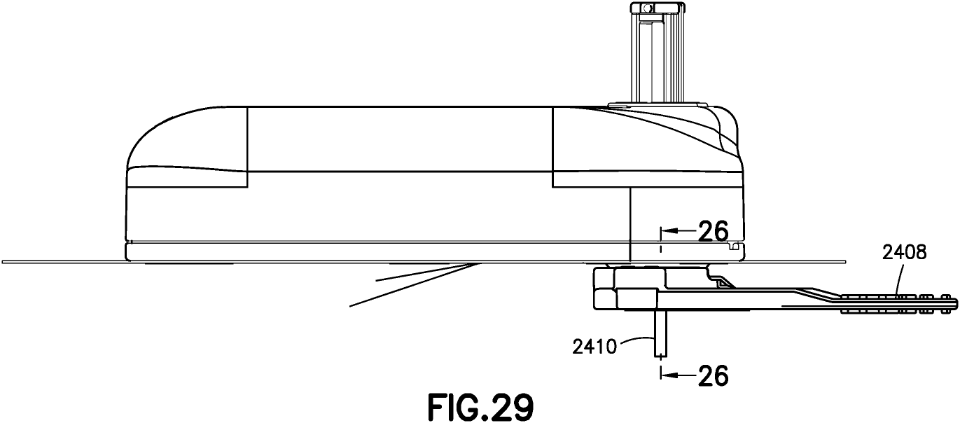

During assembly of the device 2400, fill tubing 2410 is fitted onto the unfired insertion needle 2422, and extends out of the bottom surface of the patch pump enclosure 2402. Fill tubing 2410 preferably forms a liquid tight seal around the distal end of the insertion needle. The distal portion of fill tubing 2410 extends through the fill tab 2408. FIG. 26 is a cross sectional view of fill tubing 2410 fitted onto insertion needle 2422. FIG. 27 is a bottom view of the patch pump enclosure 2402 with fill tubing 2408 extending from the bottom surface. FIG. 28 is an exploded view showing the bottom surface of the adhesive patch 2406 with fill tubing 2410 extended from the bottom surface, and with fill tab 2408 not yet attached to the patch pump enclosure 2402. During assembly, fill tab 2408 is attached to the patch pump enclosure 2402.

Fill tab 2408 includes a slot 2424 that forms a volume used for priming the device as will be described further below. FIG. 30 illustrates the fill tab 2408 assembled with the patch pump enclosure, and FIG. 31 illustrates a detailed view of the slot 2424. Slot 2424 includes barbs 2426. The distal portion of fill tubing is bent so that it is retained within slot 2424. Barbs 2426 retain the fill tubing 2410 within the slot after assembly. This enables the fill tubing 2410 to be removed with the fill tab 2408 when the device has been primed and deployed. While barbs 2426 are illustrated, any suitable method to retain the fill tubing 2410 within the slot 2424 may be used, as will be appreciated. This includes adhesive to attach the fill tubing 2410 to the inside of slot 2424, heat staking, mechanical lock, or any combination of attachment methods. Additionally, while a bent configuration of fill tubing 2410 is illustrated, the bend is not strictly necessary, and the fill tubing 2410 may instead penetrate straight through the fill tab and be attached to the fill tab using any suitable attachment method.

Once the fill tubing 2410 is inserted into the slot 2424, a hydrophobic membrane 2412 is assembled onto the adhesive patch 2406 to seal the volume within slot 2424. As a result, the catheter and insertion needle are sealed from the outside environment. Hydrophobic member 2412 provides several functions. The hydrophobic member 2412 helps prime the system by permitting air to be vented but preventing insulin from escaping. Once wetted the hydrophobic member 2412 seals the system. The hydrophobic member 2412 is also selected with properties that provide pressure relief, that is, weeping of liquid insulin, in the event of over pressure. While the hydrophobic member is illustrated and described sealed around the slot 2424 of the fill tab 2408, embodiments are not limited to this. As alternatives, the hydrophobic member 2410 may be sealed around the distal end of the fill tubing 2410, or the fill tubing itself may be made of hydrophobic material with the central opening of the fill tubing closed so that air may be vented through the fill tubing, but insulin does not penetrate the fill tubing. An exemplary material for the fill tubing and/or hydrophobic membrane is ePTFE. The term hydrophobic member as used herein will be understood to be generic to a hydrophobic membrane or a hydrophobic tubing.

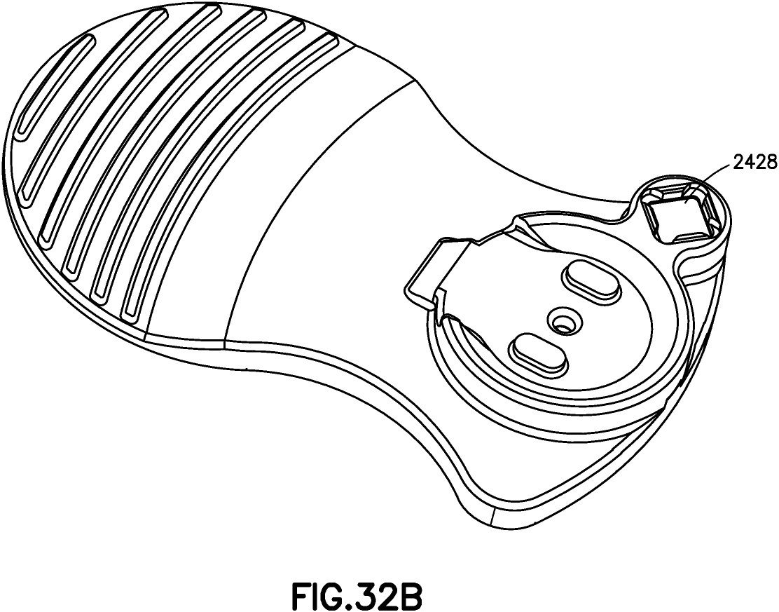

FIGS. 32A and 32B illustrate additional details of the fill tab 2408. FIG. 32A is a top view of the fill tab 2408 and FIG. 32B is a perspective view of the fill tab 2408. The fill tab 2408 according to this embodiment includes a magnet 2428. The magnet aligns with a sensor (not illustrated) provided on the printed circuit board within the patch pump enclosure 2402, such that the fill tab 2408 presence may be detected. While a magnet and magnet sensor, such as a Hall effect sensor are described as the mechanism to sense the presence of the fill tab on the patch pump enclosure, the embodiments are not so limited. Any suitable sensing mechanism could be used instead, including a photosensor, a mechanical switch, or any other sensing method.

Filling and priming of the device 2400 will now be described. The user receives the device 2400 assembled with the fill tab 2408 attached to the patch pump enclosure 2402. The user fills the reservoir with insulin via the fill port 2418. Once the reservoir is full the user primes the device 2400. Priming involves forcing insulin from the reservoir to the insertion needle and catheter via the pump. Importantly, the device 2400 is programmed to detect the presence of the fill tab 2408 during priming. Pumping is prevented during this step if the fill tab 2408 is not detected via the magnet 2428 and sensor. Otherwise, undesirable runaway flow from the reservoir to the insertion needle distal opening could occur. With the fill tab 2408 in place, the slot 2424 and hydrophobic membrane 2412 form a sealed volume that fills with insulin. The hydrophobic membrane 2412 allows air to escape. Once the volume has been filled with insulin, and air has been forced out of the volume, continued movement of the pump plunger causes the pressure within the flow channel and volume to increase. This pressure increase is detected by a pressure sensor (not shown), indicating priming has been completed, and the device 2400 is ready for deployment.

Although only a few exemplary embodiments of the present invention have been described in detail above, those skilled in the art will readily appreciate that many modifications are possible in the exemplary embodiments without materially departing from the novel teachings and advantages of this invention. Accordingly, all such modifications are intended to be included within the scope of the appended claims and their equivalents.

What is claimed is:

1. A fill closure tab for an infusion device comprising:
a gripping portion,
an attachment portion shaped to attach the fill closure tab to an infusion device, and a hydrophobic member arranged to seal a distal end of an insertion needle of the infusion device;
wherein the fill closure tab is removable from the infusion device.

2. The fill closure tab of claim 1, further comprising:
a fill assist mechanism arranged adjacent to a fill port of the infusion device.

3. The fill closure tab of claim 2, wherein the fill assist mechanism is shaped to receive a pen needle.

4. The fill closure tab of claim 2, wherein the fill assist mechanism is shaped to receive a syringe.

5. The fill closure tab of claim 1, wherein the hydrophobic member is formed of a material that changes color in the presence of medicament.

6. The fill closure tab of claim 1, wherein the hydrophobic member permits air to pass, but prevents medicament from passing through the hydrophobic member.

7. The fill closure tab of claim 1, further comprising a magnet adapted to be sensed when the fill closure tab is attached to a patch pump.

8. The fill closure tab of claim 1, further comprising a slot configured to receive a fill tube, the slot forming a volume in which the fill tube is received and at least one barb to retain the fill tube in the slot.

9. The fill closure tab of claim 8, further comprising a hydrophobic member sealed around the slot.

10. The fill closure tab of claim 1, wherein the hydrophobic member is attached to a distal end of fill tubing that seals the distal end of the insertion needle.

11. The fill closure tab of claim 1, wherein the hydrophobic member is a fill tubing sealed to a distal end of the insertion needle.

12. The fill closure tab of claim 1, wherein the hydrophobic member permits medicament to pass if a pressure of the medicament exceeds a predetermined threshold.

13. An infusion device comprising:
a housing containing a reservoir, a fill port fluidly connected to the reservoir and an insertion mechanism comprising a cannula fluidly connected to the reservoir with an insertion mechanism opening, and
a removable fill closure tab comprising:
a gripping portion,
an attachment portion shaped to attach the fill closure tab to the infusion device, and a hydrophobic membrane arranged to cover a needle opening on a bottom surface of the infusion device;
wherein the fill closure tab is removable from the infusion device.

14. The infusion device of claim 13, further comprising:
a fill assist mechanism arranged adjacent to a fill port of the infusion device.

15. The infusion device of claim 14, wherein the fill assist mechanism is shaped to receive a pen needle.

16. The infusion device of claim 14, wherein the fill assist mechanism is shaped to receive a syringe.

17. The infusion device of claim 13, wherein the hydrophobic membrane is formed of a material that changes color in the presence of medicament.

18. The infusion device of claim 13, wherein the hydrophobic membrane permits air to pass, but prevents medicament from passing through the hydrophobic membrane.

19. The infusion device of claim 13, wherein the infusion device is a patch pump.

20. The infusion device of claim 13, wherein the infusion device is an insulin pump.

21. The infusion device of claim 13, wherein the medicament is insulin.

22. The infusion device of claim 17, wherein the medicament is insulin.

23. The infusion device of claim 13, wherein the housing encloses a printed circuit board comprising a sensor configured to detect a magnet, and the removable fill closure tab comprises a magnet that aligns with the sensor when the removable fill closure tab is attached to the housing.
